(19) United States
(12) Patent Application Publication
(10) Pub. No.: US 2005/0160502 A1
(43) Pub. Date: Jul. 21, 2005

(54) Method for plant gene transfering by electrical shock and ovary injection

(75) Inventor: Pan-Chi Liou, Taichung (TW)

Correspondence Address: CHARLES E. BAXLEY, ESQ., 90 JOHN STREET, THIRD FLOOR, NEW YORK, NY, 10038, US

(73) Assignee: Taiwan Agricultural Research Institute, Council of Agriculture

(21) Appl. No.: 11/082,994

(22) Filed: Mar. 17, 2005

Related U.S. Application Data
(63) Continuation-in-part of application No. 10/228,365, filed on Aug. 27, 2002.

(51) Int. Cl. A01H 1/00; C12N 15/82

(52) U.S. Cl. 800/292

(57) ABSTRACT

A method for plant gene transfer by electrical shock and ovary injection, comprises: cutting a stigma off a style after pollination when the pollen tube reaches ovule, creating a passage between the style and ovary; injecting an exotic DNA into the ovary via the passage; inserting a conductive piece into a locule via the passage as a negative electrode; covering the ovary with a conductive layer and the conductive layer serves as a positive electrode; creating an electric field by connecting the conductive piece and the conductive layer to an electric device.

This application is a Continuation-in-Part of U.S. patent application Ser. No. 10/228,365, filed Nov. 18, 2004, to which application priority is claimed.

### FIELD OF THE INVENTION

The present invention relates to a technique about gene transfer, and more particularly, to a method for plant gene transfer by electrical shock and ovary injection.

### BACKGROUND OF THE INVENTION

A conventional way of plant gene transfer includes the following steps: (1) cloning the gene or genes from proper sources and recombining the cloned gene(s) with proper DNA vector (e. g. plasmid) used as a gene carrier; (2) transferring DNA carrier harboring the cloned gene(s) into plant cells by proper methods so as to form transgenic cell, including particle gun bombardment method, Agrobacterium-mediated method, microinjection, electroporation, virus-mediated method, and PEG method (polyethylene glycol method) etc.; (3) the above mentioned methods all require the aid of plant tissue culture to transfer the transgenic cells by way of regeneration into transgenic plants having roots, stems, and leaves; (4) examining and screening for the successful and good transgenic plants. The above mentioned methods all have their own characteristics, however, they also have some common shortcomings which are not ready for being overcome. One of the shortcomings is that the efficiency of transferring is not always satisfied. Usually a transgenic process is more likely to be successful, if a tissue culture technique with high regeneration efficiency is available. Unfortunately, in many of the cases the tissue culture technique has not yet been well established so far. This is one of the main reasons making the success of gene transfer so rare in many crops. From this point of view, a new approach of transgenic process being able to enhance transferring efficiency and not relying on the aid of tissue culture will be very valuable and useful.

### SUMMARY OF THE INVENTION

The primary object of the present invention is to provide a method for plant gene transfer by electrical shock and ovary injection that is capable of increasing the pollination rate, and including the following steps achieved (1) at a suitable time after pollination when pollen tube reaches the ovule, injecting the DNA containing exotic gene(s) into the locule of plant ovary to let DNA surround the ovule(s) inside the locule; (2) by using a controlling device to proceed an electrical shock to the ovary under the controlled voltage, current and timing, a temporary electric field is created in the ovary with negative electrode in the center and positive electrode distributing on outside around the ovary wall. The injected DNA (which carry a charge of negative electricity) will move outward from the central locule toward the ovary wall so as to increase the opportunity to enter into the ovule. This makes DNA recombination between the exotic DNA and the fertilized egg's chromosome (i.e. gene transfer) possible. Thus, the efficiency of gene transfer can be increased.

The present invention will become more obvious from the following description when connected with the accompanying drawings which show, for purposes of illustration only, a preferred embodiment in accordance with the present invention.

### DETAILED DESCRIPTION OF THE PREFERRED EMBODIMENT

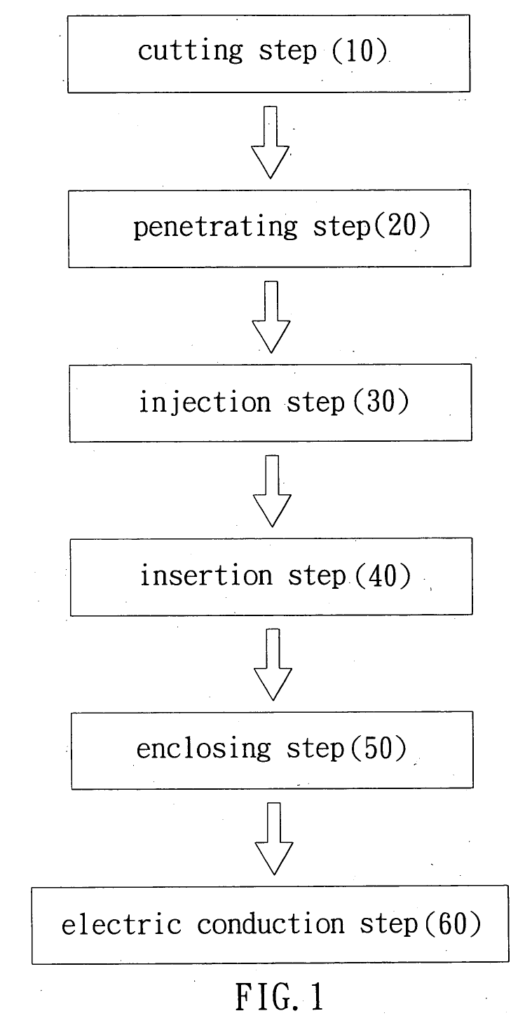

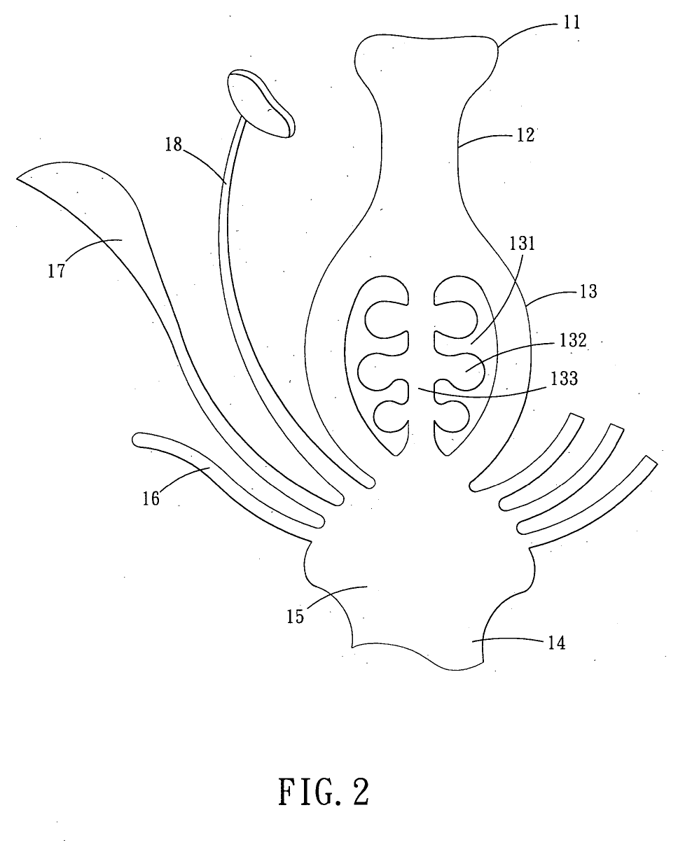

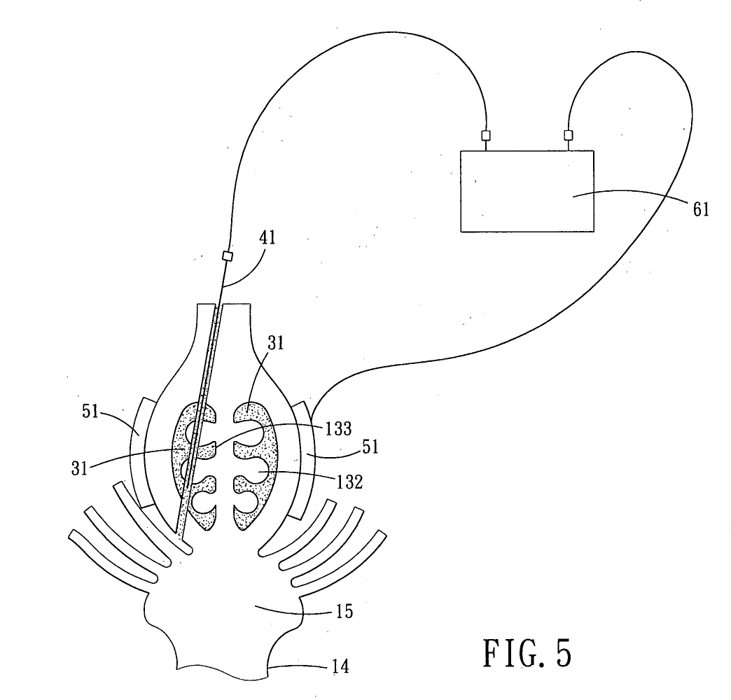

Referring to FIGS. 2 to 5, the method for plant gene transfer of the present invention comprises six steps: first: cutting step 10: cutting the stigma off at a suitable time after pollination; second: penetrating step 20: making a tiny passage or tunnel by using an injection needle to penetrate from style toward locule; third: injection step 30: injecting the DNA into the locule of ovary via the passage; fourth: insertion step 40: inserting a conductive piece into the passage so as to form a negative electrode; fifth: covering step 50: covering the ovary with conductive layer so as to form a positive electrode; sixth, electrical conduction step 60: connecting the positive and the negative electrodes to an electric device so as to create a special electric field. The six steps are operated continuously.

For the cutting step 10, referring to FIG. 2 which shows the flower of plant and includes stigma 11 (the plants employed in this embodiment are watermelon and muskmelon, but its principle is applicable to other plants), style 12, ovary 13, locule 131, ovule 132, placenta 133, floral stalk 14, receptacle 15, calyx 16, petal 17, stamens 18. The best time to cut off the stigma is after pollination of the pollen tubes when the pollen tubes stretch out from the stigma and reach the ovule(s). The cutting time can be calculated by sampling the ovary tissue at regular intervals after pollination of the pollen tubes, including ovary tissue fixing, softening, cut section, Fluorence Staining, microscope examination and the like steps, so as to figure out the development speed of the pollen tubes, and consequently to calculate the time at which the pollen tubes reach the ovules. Let's take muskmelon as an example, there are multiple ovules in its ovary, and the time for the pollen tube of the muskmelon to reach the ovules is about 8-26 hours, and the optimum time for cutting off the stigma is 24 hours after pollination.

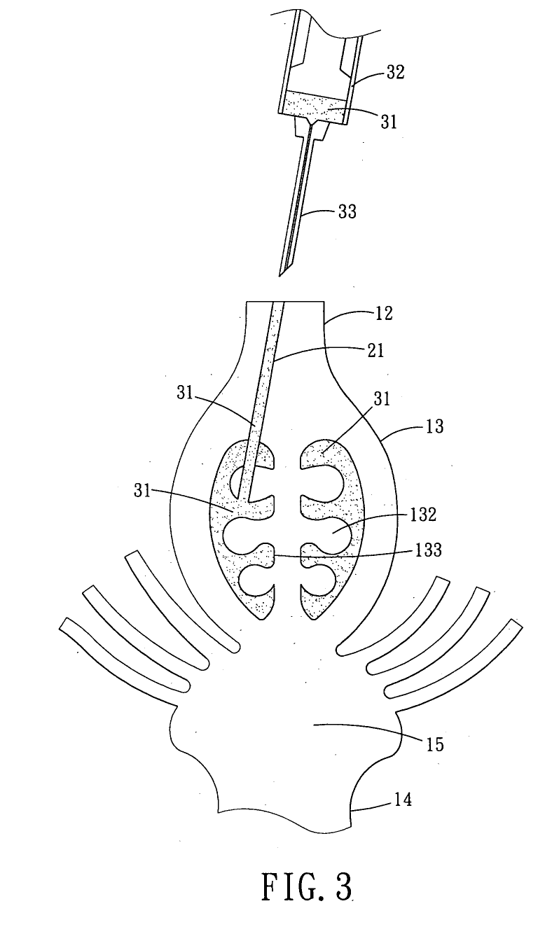

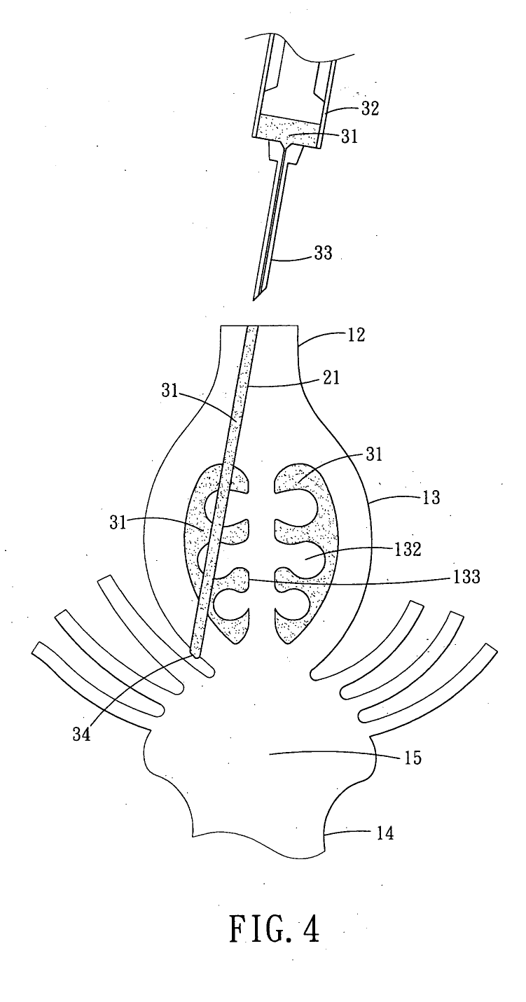

The penetrating step 20 involves defining a tiny passage 21 between the style 12 and the locule 131 by using an injection needle to penetrate into the locule 131 of the ovary 13 from the cutting place of the style 12 as shown in FIG. 3, or going further through the locule 131 to penetrate the ovary wall and make an opening 34 on it as shown in FIG. 4, and then pulling the needle out to define the tiny passage 21.

The injection step 30 involves injecting DNA 31 into the locule 131 by using a needle 33 of the injection device 32 via the passage 21, so as to make the DNA 31 surround the ovule 132. The DNA 31 is pBSGZCP DNA (13 kb) which is derived from pBI121, the DNA plasmids are transferred into coliform bacteria, and then are extracted and purified by plasmid Miniprep method. After extraction and purification, the plasmids are then stored in TE buffer, the density of the DNA 31 to be injected is 1.0 μg/μl, and the amount of the DNA 31 to be injected is 5-10 μl per flower, depending on the size of the flower to be injected. If the passage 21 is defined by penetration as shown in FIG. 4, some DNA 31 could flow out from the opening 34, but this does not affect the effect of gene transfer.

The insertion step 40 involves inserting an electrically conductive piece 51, such as a Platinum wire, into the passage 13 to form the negative electrode.

The enclosing step 50 involves covering the ovary 13 with an electrically conductive layer 51 which is used as a positive electrode.

The electrical conduction step 60 invovles connecting the positive and the negative electrodes as defined in steps 40 and 50 to a special electronic controlling device 61 so as to form a special electrical field at a certain voltage and current. Intensity of the electrical shock should be controlled appropriately, since over strong electrical shock will cause tissue death, and over weak electrical shock has no effect. The so-called intensity of electrical shock refers to an optimum combination of voltage, current, and timing. It is concluded from the experiment on the muskmelon and the watermelon that the appropriate value for voltage, current, and timing are 30-80 v, 0.1-1.0 mA and 0.1-1.0 second, respectively. Obvious, we have many options based on the scope of the above-mentioned references, i.e. 60V-0.6 mA-0.4 second, 60V-0.6 mA-0.8 second, 30V-0.6 mA-0.4 second, or 30V-0.6 mA-0.8 second. However, for any combination of the voltage, current, and timing, the testing effect of the watermelon is always not so good as that of the muskmelon, but it still has practical value. Therefore, the combination of voltage, current, and timing is best determined differently according different species of the plant.

Through the above-mentioned steps, DNA 31 is a chemical substance carrying a charge of negative electricity and will move outward in the electric field. That is to say that the DNA molecules inside locule will move from the center to the surroundings. Thus, the opportunity for DNA to enter the fertilized egg cell inside ovule 132 is increased, especially when the egg-sperm fusion (i.e. fertilization) occurs if the timing is well managed. Around the time of fertilization, some of the egg's cell wall is thin and the egg cell is somewhat similar to a protoplast. This characteristic makes the DNA 31 easy to penetrate through the cell wall, especially at the moment when fertilization occurs. As a result, the possibility that exotic DNA is combined into the chromosome of the fertilized egg, i.e. gene transfer, can be increased. The transgenic fertilized egg will then become a transgenic seed through nature development, and a transgenic plant can be produced by the transgenic seed through germination and vegetative growth. The whole procedures do not need the aid of tissue culture.

Obviously, in this method the occurrence of gene transfer depends on 5 main factors including: 1. the existence of injected DNA 31 which contains exotic gene(s); 2. sperm cells traveling toward the egg inside the extending pollen tubes after pollination; 3. egg cell(s) inside ovule(s) 132; 4. the occurrence of egg-sperm fusion (i.e. fertilization) inside ovule; and 5. electric shock treatment which increases the movement of the exotic DNA 31 inside the locule. To achieve all these factors and in order to make gene transfer occur with high possibility, an accurate timing is very important for all the steps in the method of the present invention.

The present invention can not only achieve the purpose of gene transfer, but also has the following five advantages:
1. Increasing the rate of success of plant gene transfer: the DNA is injected into the ovary at suitable time after pollination and then treated by electric shock to dramatically increase the rate of success of gene transfer.
2. Reducing the cost of the research of genetic engineering: the present invention adopts the natural way of plant breeding process to achieve the purpose of gene transfer. It is simple and easy to do. Besides, it is found that by using this method the efficiency is higher than the conventional ways used to transfer gene(s) into plants. This method does not need the aid of tissue culture to produce a complete transgenic plant from a transgenic cell. Such a characteristics is quite valuable and helpful for time and expenses saving, especially for those plant species in which the technique of tissue culture has not yet been well established.
3. Benefiting the environmental safety: The method of the present invention neither needs tissue culture technique nor relies on baterium mediation in all the procedures, therefore the problem of environmental contamination and pollution can be greatly reduced. There is also no damage to the eco-system, even the working procedures of this invention are all performed in the open field.
4. Easy to practice and perform: Using the method of the present invention, the practice and skill of gene transfer is easy to learn and teach. Once the technique for a specific plant has been well determined and established, including the timing of DNA injection and the condition of electrical shock, the experiment of gene transfer could become a routine work and can be easily performed by an assistant worker who is even without the background of genetics and molecular biology. The main part of experiment work can be done in the field or green house using a syringe of suitable size (or an injection device originally designed for gas chromatograpy analysis) and an electrical shock-inducing device. No expensive instrument and equipment is needed.
5. Particularly suitable for those plants having multiple seeds produced in one fruit, if the fruit is originated from one single pollinated flower such as tomato and watermelon: Since ovary is the basic unit to deal with in this method, this method is especially useful and valuable for those plants with multiple seeds produced in one single-flower-originated-fruit. Apparently the rate of success can be enhanced, if multiple seeds can be obtained from one single experimental treatment. The more the seeds can be obtained from one treated ovary (flower), the higher the rate of success can be expected. In agriculture, there are many crops having multiple-seeds-containing fruits which are developed from one single pollinated flower. Their species distribute in different taxonomic families, including Orchidaceae, Cucurbitaceae, Leguminosae, Solanaceae, Rosaceae, Cruciferae, Rutaceae, Myrtaceae, Liliaceae, Passifloracae, Oxalidaceae, Vitaceae, Actinidiaceae, and Caricaceae etc. We estimate that ca. ⅔ of horticultural crops are of this type, and most of these crops still lack good tissue culture research. In fact, tissue culture research even has not yet been initiated in many of these crops, and so has the genetic engineering work, therefore, the method of this invention is going to have a huge potential for application in many crops' genetic improvement.

In addition to the muskmelon and watermelon, here are also some other examples for successful gene transfer:
1, cucumber (cucumber, watermelon, muskmelon are all members of cucurbitaceae family), cutting off the stigma 8-24 hours after pollination, and injecting DNA having a concentration of 0.8 mg/ml at a volume of 15 l/flower.
2, cabbage (a member of Cruciferae family), cutting off the stigma 24 hours after pollination, and injecting DNA having a concentration of 0.3 mg/ml at a volume of 2 l/flower.
3, rice (a member of Gramineae family), cutting off the stigma 6-24 hours after pollination, and then injecting or dripping DNA having a concentration of 0.5-1.0 mg/ml at a volume of 2 l/flower.
4, cotton (a member of Bombacaceae family), cutting off the stigma 24 hours after pollination, and injecting DNA having a concentration of 0.3 mg/ml at a volume of 50 l/flower.
5, phalaenopsis orchid (a member of Orchidaceae family), cutting off the stigma 45-55 days after pollination, and injecting DNA having concentration of 0.3 mg/ml at a volume of 50 l/flower.
6, soybean (a member of Leguminosae family), cutting off the stigma 6-32 hours after pollination, and injecting DNA having concentration of 0.3 mg/ml at a volume of 10 l/flower.

While we have shown and described the embodiment in accordance with the present invention, it should be clear to those skilled in the art that further embodiments may be made without departing from the scope of the present invention.

1. A method for plant gene transfer by electrical shock and ovary injection, comprising the following steps: first, cutting a stigma off a style after pollination of pollen tube when the pollen tube stretch out from the stigma and reach ovule, second creating a passage between the style and ovary, third, injecting an exotic DNA into the ovary via the passage, fourth, inserting a conductive piece into a locule via the passage as a negative electrode, and fifth, covering the ovary with a conductive layer and the conductive layer serves as a positive electrode, sixth, creating an electric field by connecting the conductive piece and the conductive layer to an electric device, wherein the electric field provides electrical shock to the exotic DNA so as to increase chance of the exotic DNA meeting a fertilized egg, whereby to improve DNA recombination rate.

2. The method for plant gene transfer by electrical shock and ovary injection as claimed in claim 1, wherein the conductive piece is made of Platinum and used as a negative electrode, the conductive layer covering the ovary is used as a positive electrode.

3. The method for plant gene transfer by electrical shock and ovary injection as claimed in claim 2, wherein an electronic controlling device is connected to the positive electrode and the negative electrode, an electric field with negative electrode near the center of the ovary and positive electrode at the periphery of the ovary is then created by specific voltage, current and length of time, such an electric field makes the exotic DNA which carry a charge of negative electricity move outward so as to increase the opportunity of gene transfer during process of fertilization.

4. The method for plant gene transfer by electrical shock and ovary injection as claimed in claim 1, wherein the passage is defined by penetrating an injection needle from a position of the cut-off on the style toward the locule inside the ovary.

5. The method for plant gene transfer by electrical shock and ovary injection as claimed in claim 1, wherein the passage is defined by penetrating an injection needle through the ovary from the position of cut-off on the style toward the locule.

6. The method for plant gene transfer by electrical shock and ovary injection as claimed in claim 1, wherein the plant is watermelon and muskmelon, but principle of the gene transfer method is applicable to other plants.

7. The method for plant gene transfer by electrical shock and ovary injection as claimed in claim 1, wherein optimum time for cutting off the stigma is calculated by sampling ovary tissue at regular intervals after pollination of the pollen tube, including ovary tissue fixing, softening, cut section, Fluorence Staining, microscope examination and the like steps, so as to figure out development speed of the pollen tube, and consequently to calculate the time at which the pollen tube reaches the ovule.

8. The method for plant gene transfer by electrical shock and ovary injection as claimed in claim 1, wherein the exotic DNA is pBSGZCP DNA (13 kb) derived from pBI121, and the DNA 31 is injected at 1.0 μg/μl, 5-10 μl per flower, depending on the size of the flower to be injected.

9. The method for plant gene transfer by electrical shock and ovary injection as claimed in claim 1, wherein an intensity of the electrical shock refers to an optimum combination of voltage, current, and timing, and appropriate value for the voltage, the current, and the timing are 30-80 v, 0.1-1.0 mA and 0.1-1.0 second, respectively.

10. The method for plant gene transfer by electrical shock and ovary injection as claimed in claim 9, wherein intensity of the electrical shock is best determined differently according different species of the plant.
